(19) United States
(12) Patent Application Publication
(10) Pub. No.: US 2008/0233616 A1
(43) Pub. Date: Sep. 25, 2008

(54) Method for Carrying Out the Selective Evolution of Proteins in Vitro

(75) Inventor: Michael Liss, Regensburg (DE)

Correspondence Address: ROTHWELL, FIGG, ERNST & MANBECK, P.C., 1425 K STREET, N.W., SUITE 800, WASHINGTON, DC, 20005, US

(73) Assignee: GENEART AG, Regensburg (DE)

(21) Appl. No.: 12/063,196

(22) PCT Filed: Aug. 7, 2006
(86) PCT No.: PCT/EP2006/007798

§ 371 Date: Feb. 7, 2008

(30) Foreign Application Priority Data
Aug. 8, 2005 (DE) 10 2005 037 351.8

(51) Int. Cl. C12P 19/34

(52) U.S. Cl. 435/91.2

(57) ABSTRACT

The present invention relates to the production of variants of a protein in an in vitro evolution method, comprising the steps: (A) provision of an in vitro expression system comprising (i) a nucleic acid sequence S which codes for a protein Y which is to be varied, (ii) a target molecule X which is able to bind to the protein Y and/or at least one variant Y′ thereof, (iii) an RNA polymerase (Pol) which is able to transcribe the nucleic acid sequence S, (iv) a reverse transcriptase (RT) which is capable of reverse transcription of transcripts of the nucleic acid sequence S, where either the target molecule X is coupled to Pol and the protein Y is coupled to RT, or the target molecule X is coupled to RT and the protein Y is coupled to Pol, (B)incubation of the in vitro expression system from (A) under conditions which enable transcription, reverse transcription and translation to form variants Y′ of the protein Y and nucleic acid sequences S′ coding therefor, and which favor the formation of variants Y′ with improved binding properties for the target molecule X, (C) isolation and, where appropriate, characterization of those variants Y′ which exhibit improved binding properties for binding to X, and/or isolation of nucleic acid sequence variants S′ coding for Y′.

The present invention relates to the production of variants of a protein in an in vitro evolution method.

### BACKGROUND OF THE INVENTION

The increasing importance of biotechnology in the medical, chemical industry and agronomic sectors means that there is an increasing demand for proteins optimally adapted for their particular purpose of use. These proteins are initially isolated mainly from the environment, mostly within the framework of so-called metagenomic screenings. Increasingly, they are subsequently adapted by various methods to the planned “artificial” use conditions.

Thus, for example, there is a need for enzymes which are more thermally stable than their natural variants, have a different substrate specificity or show higher activities. Pharmaceutical proteins for instance are intended to have longer half-lives in order to be able to use smaller doses, or to inhibit, via the high-affinity and specific binding to target molecules, disease-associated metabolic pathways or infection routes.

### PRIOR ART

This adaptation takes place in part by rational protein design approaches. However, the present possibilities for concluding the structure of a protein from its desired function—the phenotype—and for inferring the corresponding primary sequence from the three-dimensional structure of the protein are only very limited. In order nevertheless to achieve an increase in the function of a protein, the approaches used at present are partly evolutionary and are summarized by the term “directed evolution”.

Methods of directed evolution to date are based, according to the current prior art, substantially on generating a large number of variants (progeny) of the protein to be improved, and selection thereof for improved derivatives. In this case, the number of investigated mutants may in some cases be very large, but is usually below 10¹¹. Considering a protein of only 100 amino acids, in theory 20¹⁰⁰=10¹³⁰ different variants thereof exist. A library with a size of 10¹¹ accordingly covers only a very small fraction of the possible variants. The probability of finding the theoretically best variant in such a library is approximately zero.

In order to screen a large number of variants for maximum affinity for a target molecule, a large number of protocols has already been developed (e.g. yeast two-hybrid, bacterial display, phage display, ribosomal display, mRNA display).

Screening for other properties such as, for instance, enzymatic activity mostly requires assay formants which permit the investigation of only a relatively small number of variants (<10⁶). It is common to these methods that they are confined to the generation of a library of mutants and subsequent selection thereof. Although a replication (=next generation of mutants of the “winner” of the selection which took place last) is possible manually with these protocols, it is just as complicated as the preceding step. Corresponding approaches therefore generally extend only over one to two generations. For this reason, the protocols mentioned are not evolutionary approaches in the true sense but, on the contrary, are exclusive selection of available pools of variants. Consequently, the potential for stepwise adaptation over many generations cannot be utilized, but this would be the necessary precondition for identifying in some circumstances the most active variant from an astronomically large number of possibilities.

The principle of evolution with its three preconditions—replication, mutation and selection—is capable within a given system of bringing about directed evolution from simple to highly adapted structures. This model of the so-called “blind watchmaker” enables complexity to be created without a design input and without necessary knowledge of structural data.

Bauer et al. (PNAS (1989) 86, 7937-7941) were able to show in 1989 that a continuous evolutionary process takes place in a capillary containing Qβ replicase and inoculated at one end with any RNA. A polymerization front is produced along the capillary, in the course of which the produced RNA polymers evolve as far as phage-specific sequences/secondary structures, because these are replicated more quickly by the replicase. Although this experiment exhibits no direct practical uses, it does clearly prove the potential of the three factors of replication, mutation and selection.

WO 02/22869 describes methods for use in the in vitro evolution of molecule libraries. The two hybrid system is used in this case. However, only a polymerase which has an increased error rate, but no reverse transcriptase, is used here. This document thus relates to the so-called error-prone PCR.

WO 2004/024917 describes a method for directed evolution of enzymes, where the protein and its coding DNA are spatially coupled and enclosed in a compartment. The protein is in the form of a fusion construct with a peptide tag. The starting material is a DNA library. However, no ligand-protein interactions are employed for selection, and no mutations are introduced by RNA polymerase.

WO 2005/030957 discloses an in vitro selection of proteins which are coupled as fusion proteins to coding DNA.

A similar system is also described by Bernath, K. et al. (J Mol. Biol. (Apr. 2, 2005) 345 (5), 1015-1026).

WO 01/51663 discloses integrated systems and methods for modifying nucleic acids. The NASBA method using Qβ replicase in particular is employed in this case. However, no mention of introduction of mutations by RNA polymerase is disclosed.

Although fusion proteins composed of reverse transcriptase and other proteins have been disclosed, there is no mention of the use of RT fusion proteins or T7-RNA polymerase fusion proteins in in vitro evolution.

Qβ replicase systems have also been investigated in detail (McCaskill and Bauer (Proc. Natl. Acad. Sci. USA 1993, 90, 4191-4195). This publication describes waves of evolution in a Qβ replicase system. The speed of migration of the RNA front increases with the fitness of the replicon. However, the system of the invention cannot be inferred from this publication.

WO 2004/108926 discloses the artificial evolution of proteins with improved binding ability. The proteins are encoded in RNA replicons which form, through erroneous replication, a quasi species. However, in vivo expression is involved in this case.

No in vitro systems which make it possible to evolve proteins are available to date. There is still a need for systems and methods for directed evolution of proteins with improved properties which avoids the elaborate construction and screening of mutant libraries.

The present invention relates to a method for producing variants Y′ of a protein Y, comprising the steps:

(A) provision of an in vitro expression system comprising
(i) a nucleic acid sequence S which codes for a protein Y which is to be varied,
(ii) a target molecule X which is able to bind to the protein Y and/or at least one variant Y′ thereof,
(iii) an RNA polymerase (Pol) which is able to transcribe the nucleic acid sequence S,
(iv) a reverse transcriptase (RT) which is capable of reverse transcription of transcripts of the nucleic acid sequence S, where either the target molecule X is coupled to Pol and the protein Y is coupled to RT,
   or the target molecule X is coupled to RT and the protein Y is coupled to Pol,

(B) incubation of the in vitro expression system from (A) under conditions which enable transcription, reverse transcription and translation to form variants Y′ of the protein Y and nucleic acid sequences S′ coding therefor, and which favor the formation of variants Y′ with improved binding properties for the target molecule X,

(C) isolation and, where appropriate, characterization of those variants Y′ which exhibit improved binding properties for binding to X, and/or isolation of nucleic acid sequence variants S′ coding for Y′.

The invention relates in particular to a method for producing variants Y′ of a protein Y comprising the steps:

(A) provision of an in vitro expression system comprising
(a) a nucleic acid sequence S coding for
   (a1) a transcription control sequence activatable in trans by a polymerase (Pol),
   (a2) a protein Y to be varied, and
   (a3) a reverse transcriptase (RT), or alternatively
   (a3′) a polymerase (Pol), where the sequence segments coding for (a2) and (a3) or (a3′) code for a fusion protein under the control of the transcription control sequence activatable in trans of (a1),
(b) a protein complex comprising
   (b1) a component X which is able to bind to at least one variant of the protein Y to be varied, and
   (b2) the RNA polymerase Pol for transcription of the nucleic acid sequence S from (a) or alternatively
   (b2′) the reverse transcriptase RT,

(B) incubation of the in vitro expression system from (A) under conditions which enable the formation of variants Y′ of the protein Y,

(C) isolation and, where appropriate, characterization of those variants Y′ which exhibit improved binding properties for binding to X, and/or isolation of the nucleic acid sequence variants S′ coding for Y′.

This invention thus comprises autonomous systems which on the one hand permit selection of a given variant library in the laboratory and at the same time include a replication mechanism. In this connection, the selection is intended—as a natural evolution—to achieve better-adapted variants via a preferred replication.

The present invention thus provides a method which enables evolution of proteins with improved properties, in particular with improved binding properties. The system of the invention combines an in vitro transcription with an in vitro translation and reverse transcription. It has surprisingly emerged that it is possible in an in vitro system to combine these three processes to give a natural evolution method. In this system, mRNA transcripts of a nucleic acid sequence coding for a protein to be varied are generated, which transcripts are then transcribed by reverse transcription back into cDNAs which can then be transcribed and reverse transcribed anew.

This type of amplification using RNA polymerase and reverse transcriptase is already known as alternative to PCR. One of these already known nucleic acid amplification methods is the NASBA principle (see, for example, Romana et al., 1995, J. Virol. Methods, 54 (2-3): 109-119; Romano et al., 1997, Immunol. Invest. 26 (1-2):15-28).

The reverse transcription step with reverse transcriptase which is known to have a certain error rate owing to the absence of a proofreading function preferably generates, starting from the transcripts, cDNAs of which at least some differ from the original template through mutations. Repeated transcription and reverse transcription of these cDNAs results in a large number of variants at the nucleic acid level, which code for variants of the protein to be varied.

In the in vitro system of the invention, the transcripts are translated to form proteins and variants of the originally encoded protein.

In the expression system of the invention, the reagents required for transcription, reverse transcription and translation (such as, for instance, primers, dNTPs, NTPs, tRNAs, amino acids etc.) are present in sufficient quantity in a defined space. If the nucleic acid sequence S, the target molecule X, the RNA polymerase, and the reverse transcriptase are provided at a particular site in the defined space, e.g. by inoculation, as transcription, reverse transcription and translation proceed the corresponding reagents are consumed at the inoculation site, and a progressive so-called reaction front is formed and contains the transcripts, proteins and reverse transcripts (cDNAs) which have been formed last.

The replication system used is preferably constructed in such a way that it permits an adequate mutation rate during the replications. The number of variants of an initial construct which are theoretically tested in this way is calculated from the number of progeny of a winner of one generation to the power of the total number of generations in an experiment (e.g. 10 progeny per generation with 400 generations=10⁴⁰⁰ possible variants). Although it is impossible for each of these 10⁴⁰⁰ individual variants explicitly to be present physically in one experiment, the system itself looks for a “path”, within a complex virtual terrain, which always leads upward to the absolute maximum. Only the variants along the path have existed during the experiment; all points (variants) of the terrain are theoretically possible.

The inventors of the present invention have found possibilities for controlling the formation of the protein variants in such a way that the transcription, reverse transcription and translation of particular mutated nucleic acids formed which code for protein variants with improved properties proceeds preferentially. Those cDNAs and transcripts which code for improved protein variants are thus present in larger number and advance faster at the polymerization front.

The evolutionary pressure necessary for the formation of very particular, improved variants is generated as follows.

The protein Y to be varied is able to bind to a target molecule X. Variation of the protein Y in the sense of the present invention results in variants Y′ of which at least some may have improved binding properties for the binding to the target molecule X. In order to favor the generation of such variants and where appropriate to vary these variants further in order to improve the binding properties even more, the conditions in the method of the invention are chosen so that the binding between X and the variant Y′ with improved binding properties leads to preferential reverse transcription of those transcripts which code for the variants Y′ with improved binding properties. The cDNAs resulting thereby code for variants Y′ with improved binding properties. The favoring of the reverse transcription of transcripts for variants Y′ with improved binding properties also quantitatively favors renewed transcription and translation of the variants.

In order to achieve this, there is preferably formation of a complex of RNA polymerase, reverse transcriptase, X and a variant Y′, enabling reverse transcription of the transcript by which this variant Y′ was encoded. Because of the spatial proximity of the RT to the transcript which codes for a variant Y′ with improved binding properties to X, this transcript is reverse transcribed by the same RT which is complexed with the improved variant Y′ (or forms a fusion protein therewith). FIG. 1 shows diagrammatically one method alternative according to the invention.

Y in the method of the invention is preferably encoded by a nucleic acid S as fusion protein with a protein P, where protein P is a protein which is involved in the transcription or the reverse transcription. P may thus be an RNA polymerase (Pol) or a reverse transcriptase (RT), or it may be a protein which is associated with an RNA polymerase or a reverse transcriptase or can be bound thereto.

In two preferred alternatives (1 and 2) of the method of the invention, the protein Y is either (1) associated with RT or encoded as fusion protein with RT, or (2) associated with Pol or encoded as fusion protein with Pol.

Y is preferably encoded as fusion protein by the nucleic acid sequence S. However, it may also be encoded as protein Y by the nucleic acid sequence S and, after translation, associated with the appropriate further component. This can be achieved by protein interactions or via binding molecules (e.g. biotin/avidin, biotin streptavidin etc.). Further possibilities for coupling Y or Y′ to RT or Pol include inter alia a chemical coupling via covalent linkage or else a linkage via crosslinking molecules, e.g. so-called linkers, e.g. bifunctional crosslinkers. The linking reagents suitable in this case can be selected without problems by the person skilled in the art.

In one variant for the first alternative of the method of the invention, the complex of protein Y and RT can also be encoded by two different nucleic acid sequences S1 and S2, each under the control of a suitable transcription control sequence, the result in this case not being a fusion protein but it being possible for binding between Y and RT to be brought about in another way, for example via biotin/avidin or streptavidin. This means that the nucleic acid sequence S is in this case in the form of two nucleic acid sequences S1 and S2. It is important that the protein Y to be varied is bound to an RT protein, or is in a form complexed therewith, at the end of translation.

The target molecule X is in accordance with the alternatives mentioned associated either (1) with Pol or (2) with RT, or forms a fusion protein with the respective components.

The target molecule X may be a protein, a peptide or else a nucleic acid or another molecule. It can thus be either provided as nucleic acid (e.g. encoded on a plasmid) and be expressed in the expression system of the invention, or it can be provided as molecule.

In the first alternative, a protein Y to be varied is encoded in the form of a fusion protein with a reverse transcriptase by an expression cassette. The expression cassette is provided in the form of a nucleic acid sequence S which is transcribed and translated during the evolution method of the invention. In addition to the transcription and reverse transcription, the present method permits translation of the transcript of nucleic acid S. The result thereof in the first alternative is a fusion protein which includes the protein Y to be varied, and the reverse transcriptase RT.

The reverse transcriptase can, however, also be provided according to the second alternative as complex with X, either likewise as fusion protein or as complex in which RT is coupled to X in another way. According to the second alternative, the fusion protein encoded by the nucleic acid S may, instead of RT, include the RNA polymerase Pol. In this case, an RNA polymerase must be provided at the start of the reaction. If the method is then carried out under conditions which permit transcription and translation, the polymerase Pol which is generated by translation and encoded by S can in subsequent cycles use the starting nucleic acid S and, where appropriate, variants S′ thereof as template for transcription.

The transcription control sequence is preferably a promoter which can be selected from all conventional promoters suitable for RNA polymerization reactions. The T7, T3 and SP6 RNA polymerase promoters are preferred for the purposes of the present invention. However, other promoters can also be selected.

The transcription is accordingly carried out by providing an RNA polymerase, preferably selected from T7 RNA polymerase, T3 RNA polymerase and SP6 RNA polymerase. The RNA polymerase can be encoded by a nucleic acid, or it can be introduced as protein (or fusion protein or complex) into the expression system, depending on the selected alternative of the method of the invention.

It is important for the purposes of the present invention that, if a binding occurs between X and Y or X and Y′, this binding brings the reverse transcriptase RT into the spatial proximity of the transcript. This means that a spatial complex of P/X/(Y or Y′)/RT is produced. This means that after a transcription reaction and a translation reaction have taken place, mRNA molecules which have just been transcribed and which code for Y′ are present in the spatial proximity of the RNA polymerase Pol which is coupled to the protein X. If binding between the protein X and Y or Y′ then takes place, where Y or Y′ is complexed with RT, then preferably an RT protein is located in the direct spatial proximity to the transcript. The spatial proximity between the RNA transcript and the protein RT via Y or Y′ promotes reverse transcription of the transcript for Y′.

It is thus possible to generate variants of the nucleic acid sequence S on the mRNA level, which then lead to translation products which likewise represent variants. It is possible in this way to produce variants of the protein Y to be varied, namely variants Y′. In this case, variants which bind better or which bind worse to the protein X are produced.

The evolutionary advantage for variants Y′ which have better properties in relation to binding to protein X is, in a preferred embodiment, that, shortly after transcription, a functional RT is located in the direct proximity to its own transcript, and thus for Y′, and preferably carries out reverse transcription on the latter. The result thereof is faster and/or more cDNAs which code for an improved variant Y′.

These variants with selection advantage then in turn serve as starting nucleic acid sequences S′ which can in turn be transcribed, reverse transcribed and translated. The result thereof is a preferred and thus enhanced generation of nucleic acid sequence variants S′ and variants Y′ of the protein Y, which can be isolated after an appropriate period after the method has taken place. It is also possible in the same way to make use of the error rate of the RT in the generation of variants of the reverse transcriptase.

The system of the present invention makes use of this effect by generating, through the selection of the polymerase and/or reverse transcriptase and/or the conditions, variants S′ of the originally provided nucleic acid sequence S and thus variants Y′ of the proteins encoded thereby, especially protein Y.

DNA-dependent RNA polymerases which have a particular error rate are preferably used, resulting in transcripts with mutations. These mutations may be for example point mutations, for example substitutions, deletions or insertions may be generated by the polymerase.

Both RNA polymerases and RT have no proofreading function and thus have a higher mutation rate than polymerases with proofreading function.

For this reason, therefore, the polymerases preferably used exhibit a higher error rate than the corresponding wild-type polymerases. Polymerases which have an increased mutation rate are already known in the state of the art. T7 polymerase and T3 polymerase, but also SP6 polymerase, are preferred. Variants of T7 polymerase with increased error rates already exist (Brakmann and Grzeszik, 2001, Chem. Biochem. 2, 212-219).

In the same way it is also possible to use a reverse transcriptase which is able to generate transcripts with a certain mutation rate.

Polymerases and reverse transcriptases without 5′-3′ exonuclease activity are preferably employed for Pol and RT, respectively. For the purposes of the present invention, use can be made either of the error rate of the RNA polymerase or of the reverse transcriptase, or else both.

It is also possible to increase the mutation rate (error rate) in another way to generate transcripts or/and reverse transcripts as alternative or in addition to the mentioned Pol and RT molecules with increased error rate. For example, mutagenic agents, nucleotide analogs as substrates for Pol and/or RT or/and also, for example, UV radiation can be employed. Such mutagenic substances can be selected by the skilled person because they are known in the state of the art.

The method of the invention is suitable for being carried out in vitro. The expression environment can be any expression environment suitable for this purpose, preferably using a capillary. Instead of a capillary, however, it is also possible to choose a two-dimensional expression environment, for example an environment between two glass plates. It is also possible to use a three-dimensional expression environment.

On use of a capillary, it is inoculated at one end, at the so-called inoculation region, with a nucleic acid sequence S.

If a two-dimensional system is used, it is possible to inoculate the system at a corner or else at another site, e.g. in the middle, with the nucleic acid sequence S. The degrees of freedom and the number of paths followed by the evolution of the protein Y to be varied are increased by such a two-dimensional system. The process remains controllable in both cases through observation of the polymerization fronts. This can take place for example through labeling reagents such as, for example, intercalating reagents. Novel variants are then formed as a faster front which spreads linearly in the capillary system or circularly in the two-dimensional system. Sampling or isolation of the desired variants can then take place at the end of the capillaries or at the edge of a two-dimensional system or at any other site.

It is also possible to use a large-volume three-dimensional expression system. The expression environment conditions for this purpose should then be chosen so that the reaction medium has a more viscous nature, because the liquid in a large-volume system is less stabilized by capillary forces.

The velocity of the polymerization front along the capillary or two- or three-dimensional system can serve as indicator of the progress of the development of variants and thus the improvement in the binding properties of the protein Y to be varied and its variants Y′.

In addition, all the necessary reaction conditions for transcription and translation are adjusted appropriately. In particular, these include the provision of appropriate oligonucleotides as primers, and of nucleotides, especially dNTPs, NTPs etc. Appropriate enzymes and reagents are required for the translation, such as, for instance, ribosomes, tRNAs, amino acids, energy carriers (such as, for instance, GTP and the like) etc.

Such reaction conditions can be adjusted by the skilled person without problems and the appropriate primer oligonucleotides can also be selected by the skilled person.

The method of the invention permits the automatic generation of variants Y′ of the protein Y in the provided system. It is thus possible to allow proteins Y to be varied to develop themselves in a particular direction which can be controlled through the ability of the variant Y′ to bind to the target molecule X.

It is thus possible to evolve any protein Y in the method of the invention. For this purpose, an appropriate target protein X which is able to bind to Y is simply selected, and variants with improved binding properties for X are obtained without elaborate screening.

The nucleic acid sequence variants S′ (either RNA or DNA or both) are preferably isolated at the reaction front. However, the Y′ can also be isolated.

The present invention further relates to a kit for producing a protein with improved properties, comprising

(a) an expression environment,

(b) a nucleic acid sequence S coding for an expression control sequence activatable in trans by an RNA polymerase Pol, a protein Y to be varied, a reverse transcriptase RT,

(c) a complex comprising Pol and a protein X which is able to bind to at least one variant Y′ of the protein Y to be varied, or a nucleic acid sequence coding for such a complex.

The invention further relates to an alternative kit for producing a protein with improved properties, comprising

(a) an expression environment,

(b) a nucleic acid sequence S coding for
(i) a transcription control sequence activatable in trans by an RNA polymerase Pol,
(ii) a protein Y to be varied,
(iii) an RNA polymerase Pol,

(c) a complex comprising a reverse transcriptase RT and a protein X which is able to bind to at least one variant Y′ of the protein Y to be varied, or a nucleic acid sequence coding for such a complex.

### DESCRIPTION OF THE FIGURE

FIG. 1 shows a diagrammatic representation of a method according to claim 1. A polymerization and evolution front advances in a capillary system filled with a NASBA reaction mixture. Molecules generated spatially preferentially here code for variants able to interact with the protein X coupled to T7 RNA polymerase.

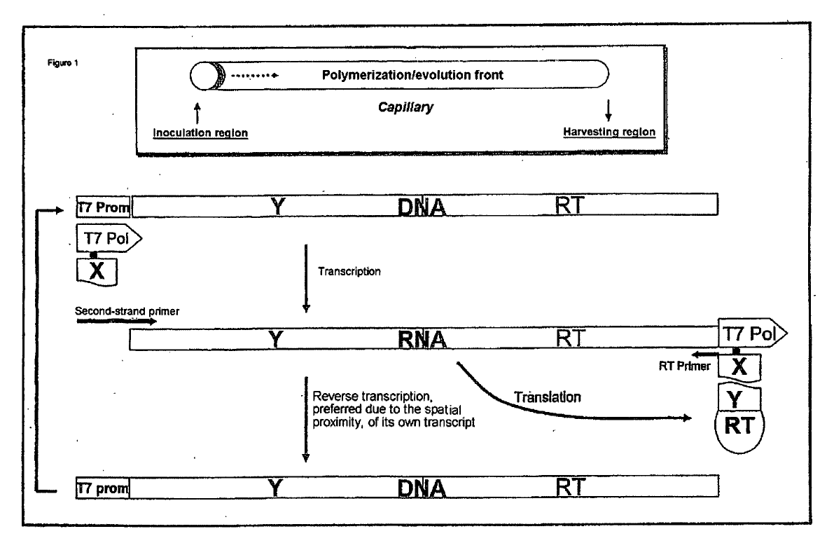

### EXAMPLE

A fusion protein composed of T7 RNA polymerase (NCBI Genbank, Acc. No. P00573) and protein A (Acc. No. CAA43604) is expressed in E. coli, purified by affinity chromatography and adjusted to a concentration of 5 μg/ml in PBS/10% glycerol.

Subsequently, the anti-HIV Env antibody 2F5 (Hofmann-Lehmann et al., 2001; Ferrantelli et al., 2003) is bound in the ratio 1:1 to the protein A domain of the chimeric fusion protein (=RNA Pol 2F5 complex).

A silanized glass capillary which is open at both ends and has an internal diameter of 1 mm and a length of 10 cm is charged with about 80 μl of the following reaction mixture.

E. coli in vitro translation reaction mixture (rapid translation system RTS) from Roche, Penzberg in the ratio 1:1 with PBS.

+RNA Pol/2F5 complex to a final concentration of 0.05 μg/ml.

+10 mg/ml PEG4000 to increase the viscosity.

+RT primer and second strand primer to a final concentration of 2 pmol/μl.

+dNTP nucleotides to a final concentration of 0.1 nmol/μl each

+NTP nucleotides to a final concentration of 0.1 nmol/μl each

+where appropriate ethidium bromide to a final concentration of 0.1 ng/μl.

The charged capillary is fixed horizontally in a chamber heated to 37° C. and inoculated at one end with 0.5 μl of a 1 μM solution of a double-stranded DNA molecule. This DNA molecule comprises the open reading frame for the fusion protein composed of HIV Env and Moloney murine leukemia virus reverse transcriptase (Acc. No. AA046154).

The reaction chamber is closed, and replication of the inoculated DNA can propagate as polymerization front alternately as transcript (RNA) and reverse transcript (DNA) along the capillary in the direction of the reagents available (to the other end).

If ethidium bromide has been added to the mixture, it is possible to establish each hour the position of the current polymerization front, and determine the end point of the reaction (end of the capillary reached), by means of a hand-held UV lamp.

After the end of the capillary is reached, 2 μl of the reaction mixture comprising the polymerization front (RNA & DNA) are removed from the capillary, and the DNA present is amplified by a PCR reaction with specific oligonucleotides. The PCR product is subcloned into a suitable vector and transformed into E. coli.

Sequence analysis of 384 clones reveals a random distribution of different sequences which code for evolved HIV Env variants having an increased affinity for the 2F5 antibody. More detailed investigations show repeating protein motifs within the variants which are responsible for the increased affinity.

### REFERENCES

(1) Bauer, G. J., J. S. McCasKill and H. Otten. 1989. Traveling waves of in vitro evolving RNA. Proc. Natl. Acad. Sci. USA. 86:7937-7941

(2) Brakmann, S. and S. Grzeszik. 2001. An Error-Prone T7 RNA Polymerase Mutant Generated by Directed Evolution. ChemBioChem. 2:212-219.

(3) Kukarin, A., M. Rong and W. T. McAllister. 2003. Exposure of T7 RNA polymerase to the isolated binding region of the promoter allows transcription from a single-stranded template. J Biol Chem. 278:2419-2424.

(4) Romano, J. W., K. G. Williams, R. N. Shurtliff, C. Ginocchio and M. Kaplan. 1997. NASBA technology: isothermal RNA amplification in qualitative and quantitative diagnostics. Immunol Invest. 26:15-28.

(5) Tanese, N., M. Roth and S. P. Goff. 1985. Expression of enzymatically active reverse transcriptase in Escherichia coli. Proc. Natl. Acad. Sci. USA. 82:4944-4948.

1. A method for producing variants Y′ of a protein Y, comprising the steps:
(A) provision of an in vitro expression system comprising
(i) a nucleic acid sequence S which codes for a protein Y which is to be varied,
(ii) a target molecule X which is able to bind to the protein Y and/or at least one variant Y′ thereof,
(iii) an RNA polymerase (Pol) which is able to transcribe the nucleic acid sequence S,
(iv) a reverse transcriptase (RT) which is capable of reverse transcription of transcripts of the nucleic acid sequence S, where either the target molecule X is coupled to Pol and the protein Y is coupled to RT, or the target molecule X is coupled to RT and the protein Y is coupled to Pol,
(B) incubation of the in vitro expression system from (A) under conditions which enable transcription, reverse transcription and translation to form variants Y′ of the protein Y and nucleic acid sequences S′ coding therefor, and which favor the formation of variants Y′ with improved binding properties for the target molecule X,
(C) isolation of those variants Y′ which exhibit improved binding properties for binding to X, and/or isolation of nucleic acid sequence variants S′ coding for Y′.

2. The method as claimed in claim 1, comprising the steps:
(A) provision of an in vitro expression system comprising
(a) a nucleic acid sequence S coding for
(a1) a transcription control sequence activatable in trans by an RNA polymerase Pol,
(a2) a protein Y to be varied, and
(a3) a reverse transcriptase (RT), or alternatively
(a3′) an RNA polymerase (Pol), where the sequence segments coding for (a2) and (a3) or (a3′) code for a fusion protein under the control of the transcription control sequence activatable in trans of (a1),
(b) a protein complex comprising
(b1) a component X which is able to bind to the protein Y and/or at least one variant Y′ of the protein Y to be varied, and
(b2) the RNA polymerase (Pol) for transcription of the nucleic acid sequence S from (a) or alternatively
(b2′) the reverse transcriptase (RT),
(B) incubation of the in vitro expression system from (A) under conditions which enable the formation of variants Y′ of the protein Y,
(C) isolation of those variants Y′ which exhibit improved binding properties for binding to X, and/or isolation of the nucleic acid sequence variants S′ coding for Y′.

3. The method as claimed in claim 1, where a complex of Pol, RT, X and a variant Y′ which enables reverse transcription of the transcript by which this variant Y′ was encoded is formed.

4. The method as claimed in claim 1, where the nucleic acid sequence S codes either for a fusion protein composed of Y and RT or for a fusion protein composed of Y and Pol.

5. The method as claimed in claim 1, where Pol, RT and/or X are encoded by a nucleic acid or are provided as proteins or fusion proteins.

6. The method as claimed in claim 1, where a capillary, a two-dimensional expression environment or a three-dimensional expression environment is used as in vitro expression system.

7. The method as claimed in claim 2, where the transcription control sequence is selected from an RNA polymerase T7 promoter, an RNA polymerase T3 promoter and an RNA polymerase SP6 promoter.

8. The method as claimed in claim 1, where RNA polymerase T7, RNA polymerase T3 or RNA polymerase SP6 is used as Pol.

9. The method as claimed in claim 1, where an RNA polymerase exhibiting an increased error rate than the corresponding wild-type polymerase is used as Pol.

10. A kit for producing a protein with improved properties, comprising
(a) an expression environment,
(b) a nucleic acid sequence S coding for
(b1) an expression control sequence activatable in trans by an RNA polymerase,
(b2) a protein Y to be varied,
(b3) a reverse transcriptase,
(c) a complex comprising an RNA polymerase and a target molecule X which is able to bind to at least one variant Y′ of the protein Y to be varied, or a nucleic acid sequence coding for such a complex.

11. A kit for producing a protein with improved properties, comprising
(a) an expression environment,
(b) a nucleic acid sequence S coding for
(b1) a transcription control sequence activatable in trans by a protein P,
(b2) a protein Y to be varied,
(b3′) an RNA polymerase,
(c) a complex comprising a reverse transcriptase and a target molecule X which is able to bind to at least one variant Y′ of the protein Y to be varied, or a nucleic acid sequence coding for such a complex.

12. The kit as claimed in claim 10, where the expression environment is selected from a capillary, a two-dimensional expression environment and a three-dimensional expression environment.

13. The kit as claimed in claim 10, additionally comprising suitable primers, dNTPs, NTPs and/or buffers.
